(19) United States
(12) Patent Application Publication
(10) Pub. No.: US 2024/0197725 A1
(43) Pub. Date: Jun. 20, 2024

(54) METHOD AND COMPOSITION FOR THE PREVENTION OR TREATMENT OF OSTEOARTHRITIS

(71) Applicant: Rhode Island Hospital, Providence, RI (US)

(72) Inventors: Wentian YANG, Pawtucket, RI (US); Lijun WANG, Providence, RI (US); Douglas C. MOORE, Barrington, RI (US)

(21) Appl. No.: 18/287,701

(22) PCT Filed: Apr. 21, 2022
(86) PCT No.: PCT/US2022/025685

§ 371 Date: Oct. 20, 2023

Related U.S. Application Data
(60) Provisional application No. 63/177,685, filed on Apr. 21, 2021.

(51) Int. Cl. A61K 31/497; A61K 45/06; A61P 19/02

(52) U.S. Cl. CPC A61K 31/497; A61K 45/06; A61P 19/02

(57) ABSTRACT

Described herein are compositions and methods for the prevention or treatment of osteoarthritis by administrating a therapeutically effective amount of at least one SHP2 antagonistic agent to the subject. The SHP2 antagonistic agent can be a SHP2 inhibitor, a PROTAC degrader of SHP2 proteins, and/or a SHP2 RNA interference or a small interfering RNA, or a combination thereof.

### STATEMENT REGARDING FEDERALLY SPONSORED RESEARCH OR DEVELOPMENT

This invention was made with government support under R01 AR066746 awarded by the National Institutes of Health. The government has certain rights in the invention.

### FIELD OF THE INVENTION

The embodiments of the present invention relate to compositions and methods for the prevention or treatment of osteoarthritis.

### BACKGROUND OF THE INVENTION

Osteoarthritis (OA) is the most common form of arthritis, affecting the hands, knees, hips, spine, and other joints. Characteristics of OA include a loss of cartilage, seen as a reduction in the joint space, and osteophytes (or bone spurs). The pain, immobility, and general disability associated with OA are familiar to most people who reach old age. OA impacts over 30 million Americans. It is the leading cause of activity limitation and absenteeism among working class adults in the United States and it is the primary cause of disability in the elderly.

Treatment currently consists of exercise and pain medications, including anti-inflammatory drugs such as NSAIDS (e.g., naproxen, ibuprofen) and, where available, joint replacement surgery, which lasts about 10-15 years on average. Hyaluronan is sometimes injected directly into the knee joint, where it is believed to help supplement the knee joint's natural synovial fluid, acting as a lubricant relieving pain and improving your ability to use the knee.

These treatments are not ideal and do not cure the condition. Surgery is not available for all joints and requires considerable recovery time as well as expense. Long-term NSAID therapy also can produce unwanted side effects. Hyaluronan injections must be repeated weekly or at least every several months and are not always successful. As such, OA treatment continues to be a clinical challenge because articular cartilage, once damaged, has limited capacity for healing.

Accordingly, there is a need in the art for OA treatments that actually repairs and/or restores the damaged cartilage.

### BRIEF SUMMARY OF THE INVENTION

The embodiments of the present invention provide a method of preventing and/or treating OA in a subject at risk of, or afflicted with, OA by administrating a therapeutically effective amount of at least one SHP2 antagonistic agent to the subject. The SHP2 antagonistic agent can be a SHP2 inhibitor, a PROTAC degrader of SHP2 proteins (SHP2 degrader), and/or a SHP2 RNA interference (RNAi) or a small interfering RNA (siRNA), or a combination thereof.

The SHP2 inhibitor can be a classic or specific inhibitor and/or an allosteric SHP2 inhibitor. In some embodiments, the SHP2 specific inhibitor is selected from PHPS1, GS-493, NSC-87877, NSC-117199, li-B08, Fumosorinone, and/or RMC-4630. In some embodiments, the SHP2 allosteric inhibitor can be SHP099, TNO-155, SHP-844, SHP-244, SHP-357, SHP-389, SHP-394, SHP-504, SHP-LY6, RMC-4550, IACS-13909, JAB-3068, JAB-3312, RLY-1971, and/or BBP-398. In some embodiments, the SHP2 degrader can be SHP-D26. In some embodiments, the SHP2 RNAi or siRNA comprises sh-Shp2-1 (sense ATATGGCGGTCCAGCATTA; SEQ ID NO:1) and sh-Shp2-2 (sense ACACTGGTGATTACTATGA; SEQ ID NO:2), complementary to nucleotides 1924 to 1942 and nucleotides 553 to 571 of SHP2 mRNA.

In some embodiments, the one or more SHP2 antagonistic agent can be provided to the subject by systemic and topical or localized administration. The delivery route can be selected from intravenous injection, intrathecal administration, oral administration, buccal administration, inhalation, nasal administration, topical administration, or locally by intraarticular injection. Administration of the one or more SHP2 antagonistic agent limits cartilage degeneration and promote cartilage regeneration and is used for OA prevention and treatment.

In some embodiments, the one or more SHP2 antagonistic agent are used to promote cartilage regeneration ex vivo and the regenerated cartilage can be subsequently administered to affected joints of the patient afflicted with OA.

Other implementations are also described and recited herein.

### BRIEF DESCRIPTION OF THE DRAWINGS

For the purpose of illustration, certain embodiments of the present invention are shown in the drawings described below. It should be understood, however, that the invention is not limited to the precise arrangements, dimensions, and instruments shown. In the drawings:

FIG. 1a-b illustrates that the Prg4 promoter is active only in articular chondrocytes and SHP2 deletion Prg4+ chondrocytes do not affect knee joint development. FIG. 1a: Knee joint fluorescent images demonstrate that Prg4+ cells primarily reside in articular cartilage. FIG. 1a,b: Prg4 promoter is inactive in the growth plate cartilage; its structure are comparable between control and SHP2 mutants.

FIG. 2a-b shows that the SHP2 deletion in Prg4+ cells increases articular cartilage thickness. H&E (FIG. 2a) and Safranin O (FIG. 2b) histologic analysis on knee joint demonstrate the structure are comparable between control and SHP2 mutants, and the thickness of articular cartilage increased in SHP2 mutants.

FIG. 3a-b shows that the SHP2 deletion in Prg4+ cells increases articular cartilage anabolic gene expression and cell proliferation. FIG. 3a: Immunostaining against SOX9 and RNA Scope against Sox9, Acan, and Col2a1 demonstrate the abundance of SOX9 protein, Acan and Col2a1 transcripts increased in the articular cartilage of SHP2KO^{Prg4ER}: R26^{ZSG} mice. FIG. 3b: EdU labeling assays in vitro show increased proliferation of SHP2 deficient PRG4+ cells compared to SHP2 competent controls.

FIG. 4 shows that the SHP2 deletion in Prg4+ cells increases articular cartilage thickness and proteoglycan content, and resists articular cartilage degeneration evoked by DMM in the tibia in SHP2KOPrg4ER mice, compared to that in SHP2CTR^{Pr94ER} mice.

FIG. 5 provides a diagrammatic representation of the pathway whereby reducing SHP2 protein expression and enzymatic activity in articular cartilage promotes anabolic gene expression and resistance to OA pathology.

FIG. 6a-f shows enhanced articular cartilage anabolism induced by SHP2D26-mediated SHP2 degradation. FIG. 6a provides diagrams depicting the general structure of a PROTAC molecule, SHP2D26, and its functional elements (bottom), as well as its functional mechanism (top). FIG. 6b provides Western blots with antibodies indicated demonstrating the efficacy and specificity of SHP2 degradation with SHP2D26 (12 hr), and the increase in SOX9, Acan1, Col2a1, Col10a1, and cell proliferation upon SHP2D26 treatment (100 nM; 48 hr) compared to vehicle (DMSO). All data from Prg4+ AC cells (n=3 replicates). FIG. 6c-d shows the viability of murine articular cartilage cells treated with concentrations ranging from 0.1-1.0 μM SHP2D26 for 48 hrs, and the reversal of SHP2D26-mediated SHP2 degradation in a week upon removal of SHP2D26. FIG. 6e shows a slight but significant increase in articular cartilage thickness (n=5) in mice that were treated with SHP2D26 (40 mg/kg, once daily, i.p for 21 days), compared to controls treated with vehicle (DMSO). In FIG. 6f, RNAscope and Immunostaining data demonstrates an increased the abundance of Col2a1 and Acan mRNA and SOX9 protein in both articular and growth plate cartilage cells in mice treated with SHP2D26, whereas Admts5 and Mmp13 or Sox9 were undetectable or comparable in the articular cartilage of both vehicle and SHP2D26 treated mice.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the terms “effective amount” and “therapeutically-effective amount” include an amount sufficient to prevent or ameliorate a manifestation of disease or medical condition, such as osteoarthritis. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of the loss of cartilage, osteophytes, and the pain, immobility, and general disability associated with OA, delay or slowing of the loss of cartilage, osteophytes, and the pain, immobility, and general disability associated with OA, and an increased mobility as compared to that expected in the absence of treatment.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively, or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Agonist: Those skilled in the art will appreciate that the term “agonist” may be used to refer to an agent (i.e., an “agonizing agent”), condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered). In some embodiments, an agonist is a binding agent that is a protein (e.g., an antibody) or a nucleic acid (e.g., an antisense oligonucleotide) that binds a target (e.g., a protein or nucleic acid) so that level, form, and/or or activity of the target is altered. In some embodiments, the altered level, form and/or activity is an increased level of altered protein expressed from the target nucleic acid sequence. Those skilled in the art, reading the present disclosure, will appreciate that, in some embodiments, an agonizing agent may bind to (and potentially agonize) a binding target, which binding causes an increase in level or activity of a further agonized target. To give a specific example, in some embodiments, an agonizing agent that binds to a nucleic acid target may alter level and/or activity of that target, and in some specific embodiments may agonize an activity of that nucleic acid target (e.g., by increasing its modification, splicing, 5′ cap formation, and/or 3′ end formation, transport, and/or translation, etc., so that a level of a desired product, e.g., mRNA, is increased) and/or may agonize a downstream target, such as a polypeptide encoded by such nucleic acid target. To give one particular such example, in some embodiments, an agonizing agent may be or comprise an oligonucleotide that binds to a primary transcript and alters its splicing pattern so that level and/or activity of a particular spliced form (e.g., mature mRNA) is increased, which may, in turn achieved increased level of a product (e.g., a polypeptide) that is or is encoded by such particular spliced form.

Antagonist: Those skilled in the art will appreciate that the term “antagonist”, as used herein, may be used to refer to an agent (i.e., an “antagonizing agent”), condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered). In some embodiments, an antagonist is binding agent that is a protein (e.g., an antibody) or a nucleic acid (e.g., an antisense oligonucleotide) that binds a target (e.g., a protein or nucleic acid) so that the level, form, and/or activity of the target is altered. In some embodiments, the altered level, form and/or activity is a decreased level of altered protein expressed from the target nucleic acid sequence. Those skilled in the art, reading the present disclosure, will appreciate that, in some embodiments, an antagonizing agent may bind to (and potentially antagonize) a binding target, which binding causes a decrease in level or activity of a further antagonized target. To give a specific example, in some embodiments, an antagonizing agent that binds to a nucleic acid target may alter level and/or activity of that target, and in some specific embodiments may antagonize an activity of that nucleic acid target (e.g., by decreasing its modification, splicing, 5′ cap formation, and/or 3′ end formation, transport, and/or translation, etc., so that a level of an undesired product, e.g., mRNA, is suppressed) and/or may antagonize a downstream target, such as a polypeptide encoded by such nucleic acid target. To give one particular such example, in some embodiment, an antagonizing agent may be or comprise an oligonucleotide that binds to a primary transcript and alters its splicing pattern so that level and/or activity of a particular spliced form (e.g., mature mRNA) is suppressed, which may, in turn achieved decreased level of a product (e.g., a polypeptide) that is or is encoded by such particular spliced form.

Antagonist Therapy: The term “antagonist therapy”, as used herein, refers to administration of an antagonist that antagonizes or inhibits a particular target of interest to achieve a desired therapeutic effect. In some embodiments, antagonist therapy involves administering a single dose of an antagonist or inhibitor. In some embodiments, antagonist therapy involves administering multiple doses of an antagonist or inhibitor. In some embodiments, antagonist therapy involves administering an antagonist or inhibitor according to a dosing regimen known or expected to achieve the therapeutic effect, for example, because such result has been established to a designated degree of statistical confidence, e.g., through administration to a relevant population. In some embodiments, antagonist therapy involves delivery of antagonizing agent as described herein. As noted above, in some embodiments, an antagonizing agent may be or comprise a binding agent that is a protein (e.g., an antibody) or a nucleic acid (e.g., an antisense oligonucleotide) that binds a target (e.g., a protein or nucleic acid) so that level, form, and/or or activity of the target is altered. In some embodiments, an antagonizing agent may bind to (and potentially agonize) a binding target, which binding causes a decrease in level or activity of a further antagonized target. To give a specific example, in some embodiments, an antagonizing agent that binds to a nucleic acid target may alter level and/or activity of that target, and in some specific embodiments may antagonize an activity of that nucleic acid target (e.g., by decreasing its modification, splicing, 5′ cap formation, and/or 3′ end formation, transport, and/or translation, etc., so that a level of a desired product, e.g., mRNA, is reduced) and/or may antagonize a downstream target, such as a polypeptide encoded by such nucleic acid target. To give one particular example, in some embodiment, an antagonizing agent may be or comprise an oligonucleotide that binds to a primary transcript and alters its splicing pattern so that level and/or activity of a particular spliced form (e.g., mature mRNA) is decreased, which may, in turn achieved decreased level of a product (e.g., a polypeptide) that is or is encoded by such particular spliced form.

Antisense: The term “antisense” is used herein to refer to a nucleic acid whose nucleotide sequence is complementary to part or all of a sequence found in a coding strand nucleic acid. Typically, a “coding strand” nucleic acid is one whose sequence includes part or all of an open reading frame or other stretch of residues that encodes part or all of a polypeptide. In some embodiments, the term “antisense” may particularly be used herein in reference to an oligonucleotide that binds specifically to a coding strand (i.e., to a target sequence within such coding strand). In some embodiments, a coding strand may include both coding and non-coding sequences (e.g., to give but one example, may be a transcript, such as a primary transcript. that includes both intron and exon sequences). Those skilled in the art, reading the present disclosure, will appreciate that, in some embodiments, an oligonucleotide may be considered or referred to as an “antisense” oligonucleotide when some or all of its sequence is complementary to non-coding portion(s) of its target strand. In some embodiments, an antisense oligonucleotide binds to coding sequences in a target sense strand; in some embodiments, an antisense oligonucleotide binds to non-coding sequences in a target coding strand. In some embodiments, an antisense oligonucleotide binds to both coding and non-coding sequences in a target coding strand. In some embodiments, an antisense oligonucleotide is characterized in that, when bound to its target sequence in a coding strand (e.g., a transcript), it alters post-transcriptional processing (e.g., one or more of modification, splicing, 5′ cap formation, and/or 3′ end formation, 5′ cap formation, and/or 3′ end formation, transport, and/or translation) of such coding strand. In some particular embodiments, an antisense oligonucleotide alters splicing of its target coding strand, Alternatively or additionally, in some embodiments, an antisense-coding strand complex is or can be degraded, e.g., by RNase H.

### Definitions for Biopharma Products/Treatments

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double- stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding a polypeptide as described herein (e.g., an antibody or antibody reagent) is comprised by a vector. In some of the aspects described herein, a nucleic acid sequence encoding a given polypeptide as described herein, or any module thereof, is operably linked to a vector. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or chimeric antigen receptor (CAR) described herein is isolated. In some embodiments, the antibody, antibody reagent, antigen-binding portion thereof, or CAR described herein is purified.

As used herein, “engineered” refers to the aspect of having been manipulated by the hand of man. For example, an antibody, antibody reagent, antigen-binding portion thereof, CAR or bispecific antibody is considered to be “engineered” when the sequence of the antibody, antibody reagent, antigen-binding portion thereof, CAR or bispecific antibody is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6, 110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragées, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically-acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher et al. (1996).¹

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid , naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;² The Encyclopedia of Molecular Cell Biology and Molecular Medicine;³ Molecular Biology and Biotechnology: a Comprehensive Desk Reference;⁴ Immunology;⁵ Janeway's Immunobiology;⁶ Lewin's Genes XI;⁷ Molecular Cloning: A Laboratory Manual.;⁸ Basic Methods in Molecular Biology;⁹ Laboratory Methods in Enzymology;¹⁰ Current Protocols in Molecular Biology (CPMB);¹¹ Current Protocols in Protein Science (CPPS);¹² and Current Protocols in Immunology (CPI).¹³

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### Osteoarthritis

Osteoarthritis (OA) is a disorder characterized by progressive joint failure in which all structures of the joint have undergone pathologic change. The essential pathologic characteristic of OA is hyaline articular cartilage loss accompanied by increasing thickness and sclerosis of the subchondral bone plate, outgrowth of osteophytes at the joint margin, stretching of the articular capsule, and weakness of the muscles bridging the joint. There are numerous pathways that lead to OA, but the initial step is often joint injury in the setting of a failure of protective mechanisms.

The earliest changes of OA may begin in cartilage. The two major components of cartilage are type 2 collagen, which provides tensile strength, and aggrecan, a proteoglycan. OA cartilage is characterized by gradual depletion of aggrecan, unfurling of the collagen matrix, and loss of type 2 collagen, which leads to increased vulnerability.

OA can affect almost any joint but usually occurs in weight-bearing and frequently used joints such as the knee, hip, spine, and hands. The hand joints that are typically affected are the distal interphalangeal (DIP), proximal interphalangeal (PIP), or first carpometacarpal (thumb base); metacarpophalangeal joint involvement is rare.

OA treatment continues to be a clinical challenge because articular cartilage, once damaged, has limited capacity for healing. Historically, cartilage was believed to be populated solely by mature chondrocytes. However, recent studies demonstrate that articular cartilage contains PRG4-expressing progenitors and their cellular derivatives “chondrocytes.”^{14,15} These Prg4+ cells and their decedents actively express the cartilage master transcription factor SOX9 and the beneficial extracellular matrix (ECM) proteins Aggrecan and COL2a1.^{16,17} Therefore, the present disclosure provides therapies that can support or promote SOX9 and anabolic ECM expression in Prg4+ cells to mitigate cartilage degeneration and improve OA treatment.

### SRC Homology Phosphatase 2

Src Homology Phosphatase 2 (SHP2) is a nonreceptor protein tyrosine phosphatase encoded by the PTPN11 gene that is expressed in cartilage cells. The present disclosure provides data indicating that SHP2 deletion or pharmacological inhibition reduces cartilage degeneration and promote cartilage cell regeneration. SHP2 inhibitors or antagonist agents that can be used in the methods of the present invention are provided in Table 1.

TABLE 1
Classic inhibitors

| | |
|---|---|
| PHPS1¹⁸ Chemical Name: 4-[2-[1,5-dihydro- 3-(4-nitrophenyl)-5-oxo-1-phenyl- 4H-pyrazol-4-ylidene]hydrazinyl]- benzenesulfonic acid sodium salt hydrate Molecular Formula: C₂₁H₁₅N₅O₆S · xNa⁺ · yH₂O Cat. No. P0039 (Sigma-Aldrich) SHP2 IC₅₀ = 2.1 μM | 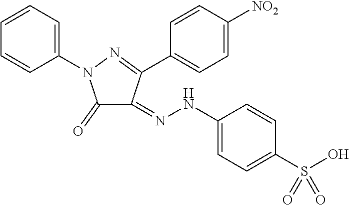 [SMILES: O=C1/C(=N/Nc2ccc(S(=O)(=O)O)cc2)C(c2ccc([N+](=O)[O-])cc2)=NN1c1ccccc1] |
| GS-493¹⁹ Chemical Name: 4-((2 Z)-2-(1,3-bis (4-Nitrophenyl)-5-oxo-1,5-dihydro- 4H-pyrazol-4-yliden)hydrazino) benzenesulfonic acid, (Z)-4-(2-(1,3- bis(4-Nitrophenyl)-5-oxo-1H-pyrazol-4(5H)- ylidene)hydrazinyl)benzenesulfonic acid Molecular Formula: C₂₁H₁₄N₆O₈S Cat. No. 5380990001 (Sigma-Aldrich) SHP2 IC₅₀ = 71 μM | 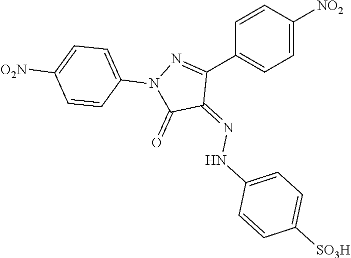 [SMILES: O=C1/C(=N\Nc2ccc(S(=O)(=O)O)cc2)C(c2ccc([N+](=O)[O-])cc2)=NN1c1ccc([N+](=O)[O-])cc1] |
| NSC-87877²⁰ Chemical Name: 8-Hydroxy-7- (6-sulfonaphthalen-2-yl)diazenyl- quinoline-5-sulfonic acid Molecular Formula: C₁₉H₁₁N₃Na₂O₇S₂ Cat. No. 565851 (Sigma-Aldrich) SHP2 IC₅₀ = 318 μM | 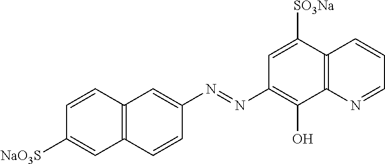 |
| NSC-117199²¹ Chemical Name: 3-[2-(2-nitrophenyl) hydrazinyl]-2-oxoindole-5-sulfonic acid Molecular Formula: C₁₄H₁₀N₄O₆S CAS No: 21303-44-6 Cat. No. bt-255107 (THE BIOTEK, Los Angeles, CA) SHP2 IC₅₀ = 47 μM²² | 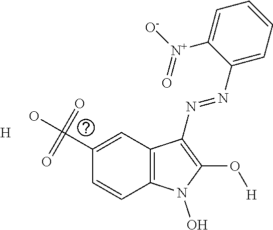 |
| Il-B08²³ SHP2 IC₅₀ = 5.5 μM²⁴ | 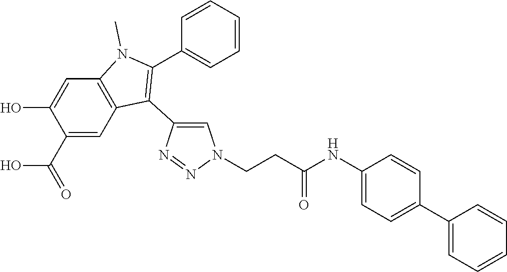 [SMILES: Cn1c(-c2ccccc2)c(-c2cn(CCC(=O)Nc3ccc(-c4ccccc4)cc3)nn2)c2cc(C(=O)O)c(O)cc21] |
| Fumosorinone (Fumos)²⁵ SHP2 IC₅₀ = 6.31 μM | 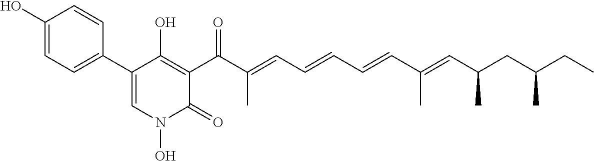 [SMILES: CC[C@@H](C)C[C@@H](C)/C=C(C)/C=C/C=C/C=C(\C)C(=O)c1c(O)c(-c2ccc(O)cc2)cn(O)c1=O] |
| RMC-4630 (aka RMC 4630; SAR 442720)²⁶ | |
| RMC-4630 is a potent and orally | |
| bioavailable small molecule that | |
| selectively inhibits the activity of SHP2. | |
| Presently in clinical trials for | |
| advanced or solid tumors, non-small cell | |
| lung cancer (NCT03989115). | |

Allosteric inhibitors

| | |
|---|---|
| SHP-099 (aka SHP 099, SHP099) Chemical Name: 6-(4-amino-4- methyl-1-piperidinyl)-3-(2,3- dichlorophenyl)-2-pyrazinamine Molecular Formula: C₁₆H₁₉Cl₂N₅ Cat. No. 20000 (Cayman Chemical, Ann Arbor, MI) SHP099 is an orally bioavailable inhibitor of SHP2, a non-receptor protein tyrosine phosphatase-SHP2 IC₅₀ = 0.071 μM.^{27,28} | 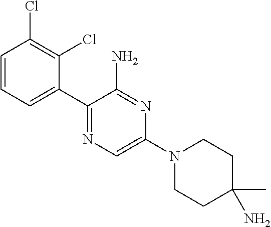 [SMILES: CC1(N)CCN(c2cnc(-c3cccc(Cl)c3Cl)c(N)n2)CC1] |
| TNO155 (aka TNO155; TNO-155; TNO 155)²⁹ Chemical Name: (3S,4S)-8-(6-amino- 5-((2-amino-3-chloropyridin-4- yl)thio)pyrazin-2-yl)-3-methyl-2- oxa-8-azaspiro[4.5]decan-4-amine Molecular Formula: C₁₈H₂₄ClN₇OS Cat. No. 207074 (MedKoo Biosciences, Inc., Morrisville, NC) TNO155 is a highly potent, selective, orally efficacious, and first-in-class SHP2 inhibitor currently in clinical trials for cancer. | 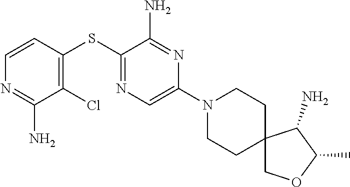 [SMILES: C[C@@H]1OCC2(CCN(c3cnc(Sc4ccnc(N)c4Cl)c(N)n3)CC2)[C@@H]1N] |
| SHP2 IC₅₀ = 0.011 μM | |
| Presently in clinical trials for adult | |
| patients with advanced solid tumors | |
| (NCT03114319). | |
| SHP844 (aka SHP 844; SHP-844)³⁰ Chemical Name: (3-chloro-4-((1- (2-hydroxy-3-methoxyphenyl)-5-oxo- [1,2,4]triazolo[4,3-a]quinazolin- 4(5H)-yl)methyl)benzoyl)-L-proline Molecular Formula: C₂₉H₂₄ClN₅O₆ Cat. No. PC-62629 (ProbeChem, Shanghai, P.R. China) SHP2 IC₅₀ = 18.9 μM | 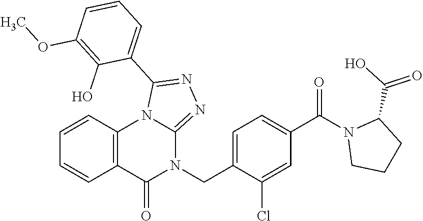 [SMILES: COc1cccc(-c2nnc3n(Cc4ccc(C(=O)N5CCC[C@H]5C(=O)O)cc4Cl)c(=O)c4ccccc4n23)c1O] |
| SHP244 (aka SHP 244; SHP-244)³¹ Chemical Name: 4-(2-Chlorobenzyl)- 1-(2-hydroxy-3-methoxyphenyl)- [1,2,4]triazolo[4,3-a]quinazolin-5(4H)-one Molecular Formula: C₂₃H₁₇ClN₄O₃ Cat. No. 563961 (MedKoo Biosciences, Inc., Morrisville, NC) SHP2 IC₅₀ = 60 μM | 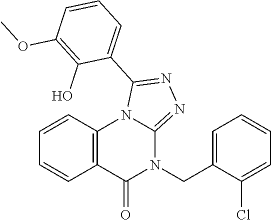 [SMILES: COc1cccc(-c2nnc3n(Cc4ccccc4Cl)c(=O)c4ccccc4n23)c1O] |
| SHP357³² SHP2 IC₅₀ = >100 μM | 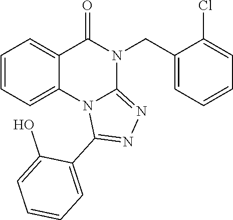 [SMILES: O=c1c2ccccc2n2c(-c3ccccc3O)nnc2n1Cc1ccccc1Cl] |
| SHP389 (aka SHP 389; SHP-389)³³ Chemical Name: 6-((3S,4S)- 4-Amino-3-methyl-2-oxa-8- azaspiro[4.5]decan-8-yl)-3-(3- chloro-2-(cyclopropylamino)pyridin-4-yl)- 5-methyl-2,5-dihydro-4H- pyrazolo[3,4-d]pyrimidin-4-one Molecular Formula: C₂₃H₂₉ClN₈O₂ Cat. No. PC-36050 (ProbeChem, | 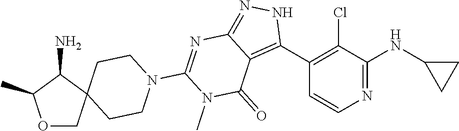 [SMILES: C[C@@H]1OCC2(CCN(c3nc4n[nH]c(-c5ccnc(NC6CC6)c5Cl)c4c(=O)n3C)CC2)[C@@H]1N] |
| Shanghai, P.R. China) | |
| SHP2 IC₅₀ = 36 μM | |
| SHP394 (aka SHP 394; SHP-394)³⁴ Chemical Name: 6-amino-2- ((3S,4S)-4-amino-3-methyl-2-oxa-8- azaspiro[4.5]decan-8-yl)-3-methyl- 5-((2-(trifluoromethyl)pyridin-3- yl)thio)pyrimidin-4(3H)-one Molecular Formula: C₂₀H₂₅F₃N₆O₂S Cat. No. HY-114397 (MedChemExpress LLC, Monmouth Junction, NJ) SHP2 IC₅₀ = 0.023 μM | 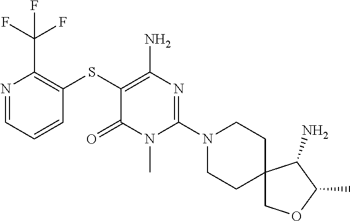 [SMILES: C[C@@H]1OCC2(CCN(c3nc(N)c(Sc4cccnc4C(F)(F)F)c(=O)n3C)CC2)[C@@H]1N] |
| SHP504³⁵ Chemical Name: 3-(4-(2- chlorobenzyl)-5-oxo-4,5-dihydro- [1,2,4]triazolo[4,3-a]quinazolin- 1-yl)-4-hydroxybenzoic acid Molecular Formula: C₂₃H₁₅ClN₄O₄ Cat. No. PC-62630 (ProbeChem, Shanghai, P.R. China) SHP2 IC₅₀ = 21 μM | 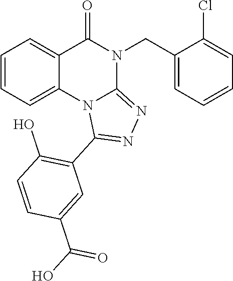 [SMILES: O=C(O)c1ccc(O)c(-c2nnc3n(Cc4ccccc4Cl)c(=O)c4ccccc4n23)c1] |
| SHP LY6 (aka LY6; SHP-LY6)³⁶ Chemical Name: (E)-1-(1-(5-(3- (2,4-dichlorophenyl)acryloyl)-2-ethoxy- 4-hydroxybenzyl)-1,2,5,6- tetrahydropyridin-3-yl)-1H-benzo[d] imidazol-2(3H)-one SHP2 IC₅₀ = 9.8 μM³⁷ | 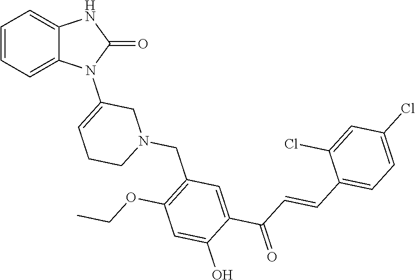 [SMILES: CCOc1cc(O)c(C(=O)/C=C/c2ccc(Cl)cc2Cl)cc1CN1CCC=C(n2c(=O)[nH]c3ccccc32)C1] |
| RMC 4550 (aka RMC-4550; RMC4550)³⁸ Chemical Name: (3-((3S,4S)-4- amino-3-methyl-2-oxa-8- azaspiro[4.5]decan-8-yl)-6-(2,3- dichlorophenyl)-5-methylpyrazin-2- yl)methanol Molecular Formula: C₂₁H₂₆Cl₂N₄O₂ Cat. No. 407910 (MedKoo Biosciences, Inc., Morrisville, NC) SHP2 IC₅₀ = 0.039 μM³⁹ | 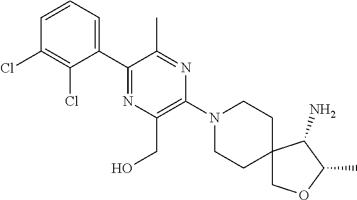 [SMILES: Cc1nc(N2CCC3(CC2)CO[C@@H](C)[C@H]3N)c(CO)nc1-c1cccc(Cl)c1Cl] |
| IACS-13909⁴⁰ Chemical Name: 1-(3-(2,3- dichlorophenyl)-1H-pyrazolo[3,4-b]pyrazin-6- yl)-4-methylpiperidin-4-amine Molecular Formula: C₁₇H₁₈Cl₂N₆ Cat. No. HY-137092 (MedChemExpress LLC, Monmouth Junction, NJ) SHP2 IC₅₀ = 0.0157 μM⁴¹ | 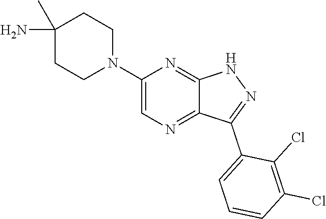 [SMILES: CC1(N)CCN(c2cnc3c(-c4cccc(Cl)c4Cl)n[nH]c3n2)CC1] |
| JAB-3068 Molecular Formula: C₂₂H₂₆F₂N₆O₂S Cat. No. CT-JAB3068 (ChemieTek, Indianapolis, IN) Presently in clinical trials for non-small cell lung cancer, head and neck cancer, esophageal cancer, and other metastatic solid tumors (NCT03518554). | 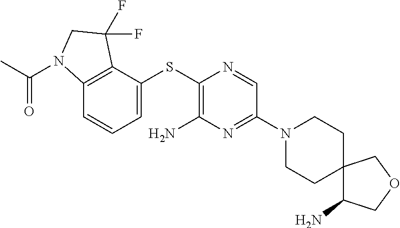 [SMILES: CC(=O)N1CC(F)(F)c2c(Sc3ncc(N4CCC5(CC4)COC[C@H]5N)nc3N)cccc21] |
| JAB-3312 | |
| Molecular Formula: C₂₂H₂₆F₂N₆O₂S | |
| Cat. No. CT-JAB3068 (ChemieTek, | |
| Indianapolis, IN) | |
| Presently in clinical trials for non- | |
| small cell lung cancer, colorectal | |
| cancer, pancreatic ductal carcinoma, | |
| esophageal squamous cell | |
| carcinoma, head and neck cancer, | |
| breast cancer, and other solid | |
| tumors (NCT04045496). | |
| RLY-1971 | |
| RLY-1971 is designed to be an oral, | |
| small molecule, potent and | |
| selective inhibitor of the protein | |
| tyrosine phosphatase SHP2 that binds | |
| and stabilizes SHP2 in its inactive | |
| conformation. Presently in clinical | |
| trials for advanced or metastatic solid | |
| tumors (NCT04252339). | |
| BBP-398 (aka BBP 398; IACS-13909; IACS-15509)⁴² Chemical Name: 4-Piperidinamine, 1-[3-(2,3-dichlorophenyl)-1H- pyrazolo[3,4-b]pyrazin-6-yl]-4-methyl- Molecular Formula: C₁₇H₁₈Cl₂N₆ Cat. No. No. S9703 (SeleckChem.com, distributed by ThermoFisher, Pittsburgh, PA) BBP-398 is a specific and potent allosteric inhibitor of SHP2 that suppresses signaling through the MAPK pathway. Presently in clinical trials for advanced solid tumors (NCT04528836). | 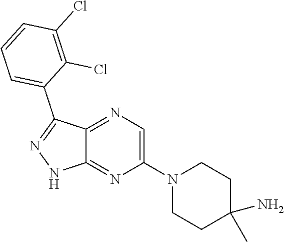 [SMILES: CC1(N)CCN(c2cnc3c(-c4cccc(Cl)c4Cl)n[nH]c3n2)CC1] |

Proteolysis-Targeting Chimera (PROTAC) Degrader of SHP2 Proteins

| | |
|---|---|
| SHP-D26 (aka SHP2-D26; SHP2D26)⁴³ Chemical Name: (2S,4R)-1-((S)- 2-(9-(4-(4-((3-((3-amino-5-(4-amino-4- methylpiperidin-1-yl)pyrazin-2- yl)thio)-2-chlorophenyl)amino)-4- oxobutanoyl)piperazin-1-yl) nonanamido)-3,3-dimethylbutanoyl)-4- hydroxy-N-((S)-1-(4-(4-methylthiazol- 5-yl)phenyl)ethyl)pyrrolidine-2- carboxamide Molecular Formula: C₅₆H₇₉Cl₁₂N₁₂O₆ S₂ Cat. No. 462070 (MedKoo Biosciences, Inc., Morrisville, NC) SHP2 DC₅₀ = 0.0026-0.006 μM SHP2-D26 is a first, potent, and effective PROTAC degrader of SHP2 protein. SHP2-D26 achieves DC₅₀ values of 6.0 and 2.6 nM in esophageal cancer KYSE520 and acute myeloid leukemia MV4; 11 cells, respectively, and can | 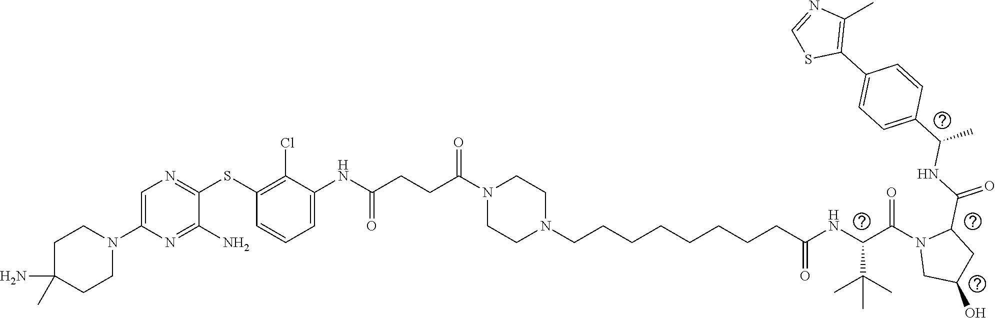 [SMILES: Cc1ncsc1-c1ccc([C@H](C)NC(=O)C2C[C@@H](O)CN2C(=O)[C@@H](NC(=O)CCCCCCCCN2CCN(C(=O)CCC(=O)Nc3cccc(Sc4ncc(N5CCC(C)(N)CC5)nc4N)c3Cl)CC2)C(C)(C)C)cc1] |
| reduce SHP2 protein levels by >95% | |
| in cancer cells. SHP2-D26 is >30-times | |
| more potent in inhibition of phosphorylation | |
| of extracellular signal-regulated | |
| kinase (ERK) and of cell growth than SHP099. | |

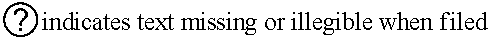

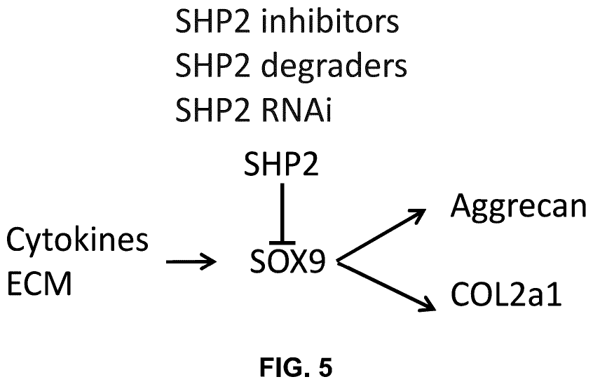

The present disclosure provides a method of preventing and/or treating OA in a subject at risk of, or afflicted with, OA by administrating a therapeutically effective amount of at least one SHP2 antagonistic agent to the subject. As depicted in FIG. 5, the SHP2 antagonistic agent can be a SHP2 inhibitor, a PROTAC degrader of SHP2 proteins SHP2 (SHP2 degrader), and/or a SHP2 RNA interference (RNAi) or a small interfering RNA (siRNA).

The SHP2 inhibitor can be a classic or specific inhibitor or an allosteric SHP2 inhibitor. The SHP2 specific inhibitor can be PHPS1, GS-493, NSC-87877, NSC-117199, li-B08, Fumosorinone, and/or RMC-4630. The SHP2 allosteric inhibitor can be SHP099, TNO-155, SHP-844, SHP-244, SHP-357, SHP-389, SHP-394, SHP-504, SHP-LY6, RMC-4550, IACS-13909, JAB-3068, JAB-3312, RLY-1971, and/or BBP-398. The SHP2 degrader can be SHP-D26. The SHP2 RNAi or siRNA comprises
sh-Shp2-1 (sense ATATGGCGGTCCAGCATTA; SEQ ID NO:1) and
sh-Shp2-2 (sense ACACTGGTGATTACTATGA; SEQ ID NO:2),
   complementary to nucleotides 1924 to 1942 and nucleotides 553 to 571 of SHP2 mRNA).

In some embodiments, the one or more SHP2 antagonistic agent can be provided to the subject by systemic and topical or localized administration. The delivery route can be selected from intravenous injection, intrathecal administration, oral administration, buccal administration, inhalation, nasal administration, topical administration, or locally by intraarticular injection. Administration of the one or more SHP2 antagonistic agent limits cartilage degeneration and promote cartilage regeneration and is used for OA prevention and treatment.

In some embodiments, the one or more SHP2 antagonistic agent are used to promote cartilage regeneration ex vivo and the regenerated cartilage can be subsequently administered to affected joints of the patient afflicted with OA.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 Role of SHP2 Deficiency in PRG4+ Articular Cartilage Cells in Cartilage Cell Proliferation

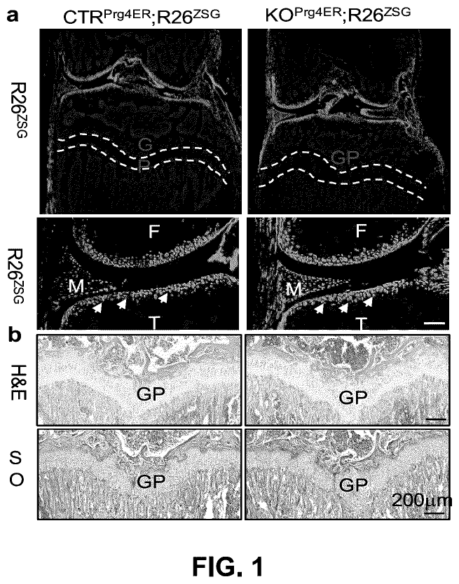

The role of SHP2 in cartilage cell proliferation was examined by studying genetically modified mice that express R26ZsG green-fluorescent reporter (R26^{ZsG}) and lack SHP2 in PRG4+ cartilage progenitors and chondrocytes (green). As shown in FIG. 1, the Prg4 promoter is active only in articular chondrocytes and SHP2 deletion in Prg4+ chondrocytes does not affect knee joint development. The knee joint fluorescent images in FIG. 1a demonstrate that Prg4+ cells primarily reside in articularcular cartilage. The Prg4 promoter is inactive in the growth plate cartilage; its structure is comparable between control and SHP2 mutants (FIG. 1a and FIG. 1b).

### Materials and Methods

Transgenic mice: Ptpn11 floxed (Ptpn11^{fl}),⁴⁴ Tg(Prg4-CreER),⁴⁵ and Tg(Rosa26^{ZSG})⁴⁶ mice were reported previously. Mice bearing a Ptpn11^{fl} allele were interbred to Tg(Prg4-CreER) and Tg(Rosa26^{ZSG}) mice to generate SHP2CTR^{Prg4ER}; R26^{ZSG} and SHP2KO^{Prg4ER}; R26^{ZSG} mice. To delete SHP2 in PRG4+ articular chondrocytes (ACs) tamoxifen was dissolved in DMSO-ethanol-corn oil mixture at a concentration of 10 mg/mL and injected intraperitoneally (1 mg/per mouse/each dose). Control and SHP2 mutant animals were sacrificed at the indicated time points and used for X-ray, μ-CT, histological, biochemical and biological analyses. All transgenic mice were maintained on C57BL/6 background and studied in accordance with the Institutional Animal Care and Use Committee approved protocols.

Histology: To examine the effect of SHP2 deficiency on gross knee joint development, mice were administered three doses of TM at week 2 and sacrificed at week 6 and 10. All specimens were fixed in 4% paraformaldehyde (PFA) overnight or 2-3 days at 4° C. For hematoxylin and eosin (H&E) and Safranin O staining to view the gross histology, fixed skeletons were decalcified before sectioning. For immunostaining with antibodies indicated, 7 μM of knee joint frozen sections were used. To trace the fate of Prg4-expressing ACs in vivo, knee joint frozen sections were examined microscopically at indicated time points. DAPI was used to counterstain the nucleus. All fluorescent and phase contrast images were obtained using a Nikon digital fluorescence microscope and an Aperio slide scanner (Vista, CA). Immunostaining of tissue sections was visualized using Texas red-labeled secondary antibody following the manufacturer's instructions.

RNAscope: Knee joint frozen sections (7 μm) from mice were used for in situ hybridization with probes against murine Sox9, Acan, and Col2a1. Hybridization and detection of hybridization signals were achieved using RNAScope® HD-Brown kit per the manufacture's instruction (Advanced Cell Diagnostics) and evaluated using NIH ImageJ software. Cell proliferation was carried out using EdU labeling assays.

### Results

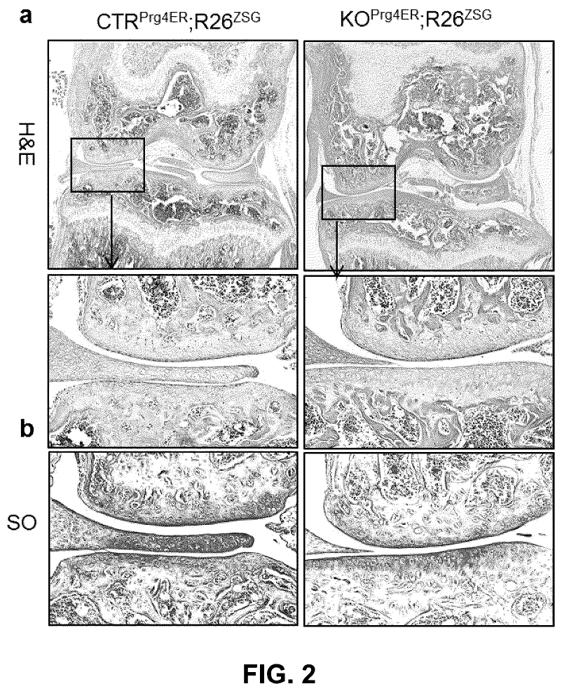

SHP2 deficiency in PRG4+ articular cartilage cells promoted cartilage cell proliferation and increased the thickness of articular cartilage by 39%. As shown in FIG. 2, HE(a) and Safranin O(b) histologic analysis on knee joint demonstrated that the structure are comparable between control and SHP2 mutants, and the thickness of articular cartilage increased in SHP2 mutants. The thickness for SHP2CTR^{Prg4} was 51.76 μm±3.29 compared to 71.82 μm±10.17 for SHP2KO^{Prg4} (Students' t-test, P<0.05).

Prg4+ cells and their decedents actively express the cartilage master transcription factor SOX9 and the beneficial extracellular matrix (ECM) proteins Aggrecan and COL2a1.^{47,48} SOX-9, the cartilage master transcription factor, is expressed by proliferating but not hypertrophic chondrocytes that is essential for differentiation of precursor cells into chondrocytes.⁴⁹ Aggrecan is the major proteoglycan in the articular cartilage. This molecule is important in the proper functioning of articular cartilage because it provides a hydrated gel structure (via its interaction with hyaluronan and link protein) that endows the cartilage with load-bearing properties.⁵⁰ Collagen, type II, alpha 1 (also known as COL2a1) is the major component of the cartilage matrix, and together with other proteins and proteoglycans, COL2A1 can form complex extracellular scaffolds to bear mechanical forces, maintain physiological homeostasis, and provide anchoring sites for chondrocytes, extracellular matrix (ECM) molecules, and growth factors.⁵¹ Cartilage cell proliferation was assessed by measuring expression of SOX9 protein and Aggrecan and COL2a1 transcripts.

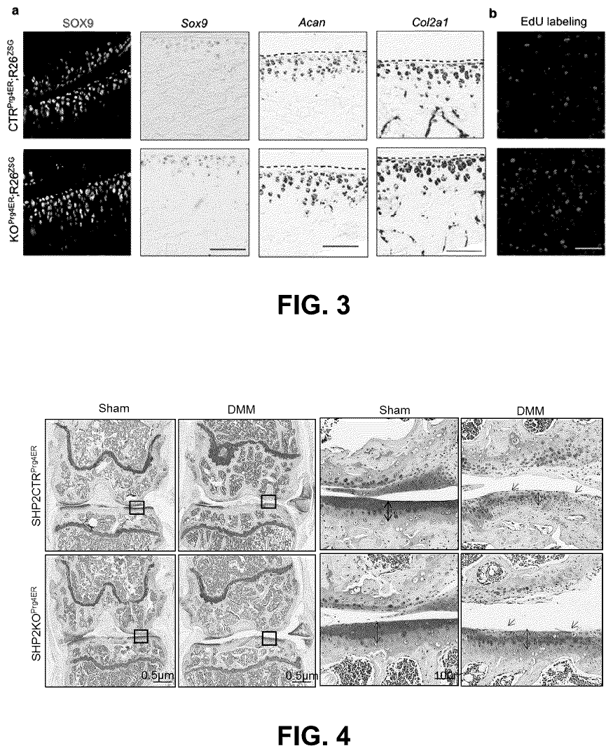

As shown in FIG. 3, SHP2 deficiency in PRG4+ articular cartilage cells resulted in an elevated expression of SOX9 protein and increases the abundance of Aggrecan and COL2a1 transcripts without any apparent effect on joint development and function. For the SOX9-expressing cells in the articular cartilage, its number increased from 45% (mean) in SHP2CTR^{Prg4} mice to 64% (mean in SHP2KO^{Pr94} mice (n=3, p<0.05, statistically significant); SOX9 level increased 31% in articular chondrocytes when SHP2 was knocked down using shRNA.⁵² For the Aggrecan transcript, the Acan relative expression was 1±0.21 in SHP2CTR^{Prg4} compared to 1.91±0.44 in SHP2KO^{Pr94}, representing a statistically significant increase (Students' t-test, P<0.05). The Col2a1 relative expression was 1±0.02 in SHP2CTR^{Prg4} compared to 1.32±0.13 in SHP2KO^{Prg4}, representing a statistically significant increase (Students' t-test, P<0.05).

Together, these data strongly suggest that SHP2 deletion or pharmacological inhibition can reduce cartilage degeneration and promote cartilage cell regeneration.

### Example 2 Effects of Attenuation of Reduction Of Shp2 Protein Expression in Articular Cartilage on Chondrocyte Proliferation, SOX9 Expression, Ecm Protein Production, and the Mitigation of Osteoarthritis Pathology

The present study assessed the ability of mitigating OA pathology by reducing SHP2 protein expression in articular cartilage.

### Materials and Methods

### Animals

To test whether SHP2 blockage in cartilage cells has a positive effect on cartilage, male and female mice that either express (control; SHP2CTR^{Prg4ER}; R26^{ZSG}) or lack SHP2 (knockout; SHP2KO^{Prg4ER}; R26^{ZSG}) in PRG4+ articular cartilage cells were generated using “Cre-IoxP” technology. R26^{ZSG} was bred into both strains for lineage tracing.

At 10 weeks of age, the mice underwent surgery to destabilize the medial meniscus (DMM) on the left knee, which is a validated method of producing an animal model of OA.⁵³ Sham surgery was performed on the right knee to provide within-animal controls. All mice were sacrificed at 22 weeks of age, their knee joints were collected, fixed, and processed to assess the effect of SHP2 deletion on OA pathology.

### Results

As shown in FIG. 4, articular cartilage thickness increased in the SHP2KO^{Prg4ER}. R26^{ZSG} mice when compared to SHP2CTR^{Prg4ER}; R26^{ZSG} mice in the sham group. Most importantly, OA pathology was significantly mitigated in the SHP2KO^{Pr94ER}; R26^{ZSG} mice, featuring a marked retention of articular cartilage. In addition to increasing articular cartilage thickness, SHP2 deletion in Prg4+ cells induced an increased proteoglycan content and resisted articular cartilage degeneration evoked by DMM in the tibia in SHP2KO^{Prg4}ER mice, compared to that in SHP2CTR^{Prg4ER} mice.

Collectively, these data strongly suggest that reducing SHP2 protein expression in articular cartilage resulted in an enhanced chondrocyte proliferation, SOX9 expression, ECM protein production, and a mitigation of OA pathology.

### Example 3 Effects of Attenuation of SHP2 Enzymatic Activity in Articular Cartilage on Chondrocyte Proliferation, SOX9 Expression, ECM Protein Production, and the Mitigation of Osteoarthritis Pathology

The present study assesses the ability of mitigating OA pathology by reducing SHP2 enzymatic activity in articular cartilage using a SHP2 inhibitor.

To test whether the inhibition of SHP2 enzymatic activity in cartilage cells has a positive effect on cartilage, male and female mice that express SHP2 (SHP2CTR^{Prg4ER}; R26^{ZSG}) in PRG4+ articular cartilage cells are used.

At 10 weeks of age, the mice undergo surgery to destabilize the medial meniscus (DMM) on the left knee, which is a validated method of producing an animal model of OA. 54 Sham surgery is performed on the right knee to provide within-animal controls. Mice are randomly separated into two groups: Control and Treatment. Mice in the Control group receive a daily intraarticular administration of the vehicle from week-10 to week-22 whereas mice in the Treatment group receive a daily intraarticular administration of an effective amount of a SHP2 inhibitor from Table 1 from week-10 to week-22. At 22 weeks of age, all mice are sacrificed, and their knee joints are collected, fixed, and processed to assess the effect of SHP2 deletion on OA pathology as described above in Example 2.

Mice in the Treatment group display an increased articular cartilage thickness compared to the Control group. Most importantly, OA pathology is significantly mitigated in the Treatment group, featuring a marked retention of articular cartilage compared to mice in the Control group. In addition to increasing articular cartilage thickness, SHP2 inhibition induces an increased proteoglycan content and decreased articular cartilage degeneration evoked by DMM in the tibia in the Treatment group mice, compared to that in the Control group mice.

Collectively, these data strongly suggest that reducing SHP2 enzymatic activity in articular cartilage using a SHP2 inhibitor results in an enhanced chondrocyte proliferation, SOX9 expression, ECM protein production, and a mitigation of OA pathology.

### Example 4 Enhanced Articular Cartilage Anabolism Induced by Protac-Mediated SHP2 Degradation

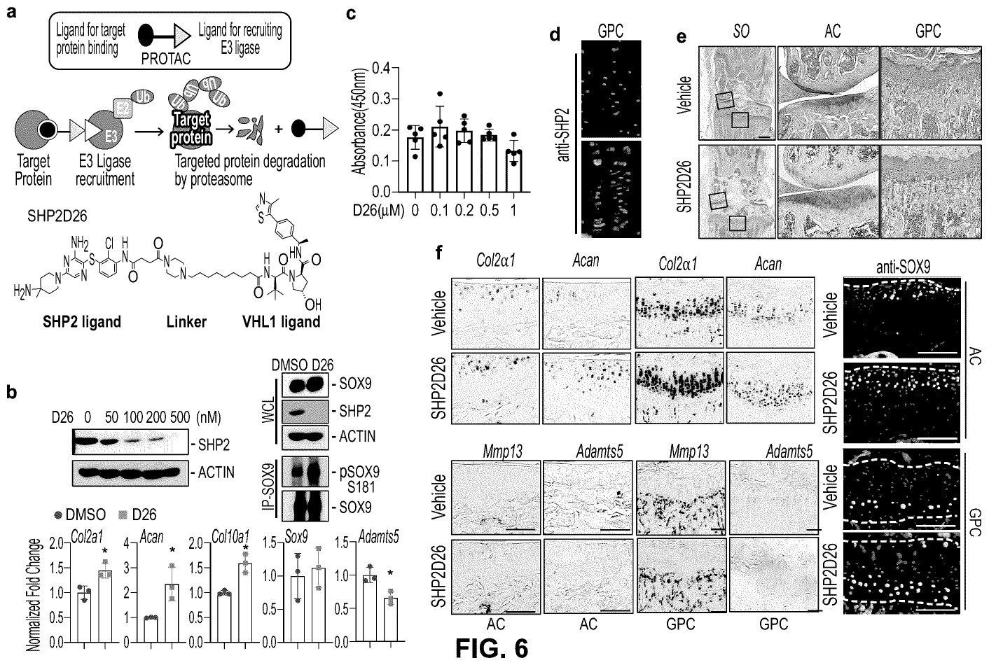

The previous Examples described the chondroprotective role of SHP2 depletion in cartilage. PROTACs are heterobifunctional small molecules designed to remove target proteins via selective intracellular proteolysis for potentially targeted therapy.⁵⁵ PROTACs juxtapose E3 ubiquitin ligase with a target protein, leading to target protein ubiquitination and subsequent degradation by the proteasome. See, FIG. 6a top panel. The present study assessed the ability of PROTAC SHP2 degraders to induce articular cartilage anabolism.

SHP2-D26 (aka SHP2D26, D26; see, FIG. 6a bottom panel) was used in the present study, as it has been reported to be a potent and effective PROTAC degrader of the SHP2 protein.⁵⁶ It has been shown to degrade SHP2 in esophageal cancer cells by over 95% at 20 nM (12 hrs) and being over 30-times more effective at inhibiting ERK than the SHP2 allosteric inhibitor, SHP09931.⁵⁷

### Assessment of SHP2D26 Efficacy and Toxicity in Cartilage Cells

First, the toxicity and efficacy of SHP2D26 were tested in cartilage cells. As shown in FIG. 6b, SHP2D26 essentially eliminated SHP2 at 50 nM (12hrs) in Prg4+ murine articular cartilage (AC) cells, with corresponding increases in SOX9, Acan1, Col2a1, Col10a1, and cell proliferation.

With respect to toxicity, murine articular cartilage cells were treated with indicated concentration of SHP2D26 for 48 hrs. Cell viabilities were measured using WST1 (n=3). As shown in FIG. 6c, treatment of Prg4+ AC cells with SHP2D26 at concentrations as high as 1 μM did not compromise cell viability, indicating that SHP2D26 is very tolerable by articular cartilage cells. Moreover, as shown in FIG. 6d, SHP2D26-mediated SHP2 degradation was shown to be reversable in a week; about 50% of SHP2 was recovered in chondrocytes after the complete withdrawal of SHP2D26 from the culture medium.

### Assessment of the Ability of SHP2-D26 to Induce Anabolism in Cartilage Cells

In order to examine the impact of SHP2D26 treatment on AC histology and the expression of anabolic and catabolic gene expression in vivo, adult nude mice were treated daily with vehicle (DMSO) or 40 mg/kg SHP2-D26 (i.p.) for 21 days. The mice were then sacrificed to harvest the knee joints, which were then fixed for 4 days in formalin, decalcified, and sectioned for Safranin O staining.

As shown in FIG. 6e, Safranin O staining revealed a slight but significant increase in articular cartilage thickness (n=5) in knee joints of mice that were treated with SHP2D26 compared to vehicle-treated controls.

Consistent with our previous findings, SHP2D26 treatment also increased the abundance of Col2a1, Acan and SOX9 in knee joint frozen sections; Admts5 and Mmp 13 or Sox9 were undetectable or comparable in the AC of both vehicle and SHP2D26 treated mice. See, FIG. 6f.

In conclusion, these data strongly suggest that a PROTAC degrader of SHP2 proteins (i.e., a SHP2 degrader) effectively degrade SHP2 in chondrocytes, and SHP2 treatment promotes chondrogenic gene expression and articular cartilage anabolism.

### References

¹ Isselbacher, et al. (1996). HARRISON'S PRINCIPLES OF INTERNAL MEDICINE, 13 ed., 1814-1882.
² THE MERCK MANUAL OF DIAGNOSIS AND THERAPY, (2011). 19th Edition, published by Merck Sharp & Dohme Corp., (ISBN 978-0-911910-19-3).
³ THE ENCYCLOPEDIA OF MOLECULAR CELL BIOLOGY AND MOLECULAR MEDICINE, Robert S. Porter et al. (eds.), published by Blackwell Science Ltd., 1999-2012 (ISBN 9783527600908).
⁴ MOLECULAR BIOLOGY AND BIOTECHNOLOGY: A COMPREHENSIVE DESK REFERENCE, (1995). Robert A. Meyers (ed.), published by VCH Publishers, Inc. (ISBN 1-56081-569-8).
⁵ IMMUNOLOGY, (2006). Werner Luttmann, published by Elsevier.
⁶ JANEWAY'S IMMUNOBIOLOGY, (2014). Kenneth Murphy, Allan Mowat, Casey Weaver (eds.), Taylor & Francis Limited, (ISBN 0815345305, 9780815345305).
⁷ LEWIN'S GENES XI, (2014). published by Jones & Bartlett Publishers (ISBN-1449659055).
⁸ Michael Richard Green and Joseph Sambrook, (2012). MOLECULAR CLONING: A LABORATORY MANUAL, 4th ed., Cold Spring Harbor Laboratory Press, Cold Spring Harbor, N.Y., USA (ISBN 1936113414).
⁹ Davis et al., (2012). BASIC METHODS IN MOLECULAR BIOLOGY, Elsevier Science Publishing, Inc., New York, USA (ISBN 044460149X).
¹⁰ LABORATORY METHODS IN ENZYMOLOGY: DNA, (2013). Jon Lorsch (ed.) Elsevier (ISBN 0124199542).
¹¹ CURRENT PROTOCOLS IN MOLECULAR BIOLOGY (CPMB), (2014). Frederick M. Ausubel (ed.), John Wiley and Sons (ISBN 047150338X, 9780471503385).
¹² CURRENT PROTOCOLS IN PROTEIN SCIENCE (CPPS), (2005). John E. Coligan (ed.), John Wiley and Sons, Inc.
¹³ CURRENT PROTOCOLS IN IMMUNOLOGY (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
¹⁴ Kozhemyakina, E., et al. (2015). “Identification of a Prg4-expressing articular cartilage progenitor cell population in mice.” Arthritis Rheumatol. 67: 1261-1273.
¹⁵ Decker, R. S., et al. (2017). “Cell origin, volume and arrangement are drivers of articular cartilage formation, morphogenesis and response to injury in mouse limbs.” Dev. Biol. 426: 56-68.
¹⁶ Haseeb, A., et al. (2021). “SOX9 keeps growth plates and articular cartilage healthy by inhibiting chondrocyte dedifferentiation/osteoblastic redifferentiation.” Proc. Natl. Acad. Sci. USA 118(8): e2019152118; DOI: 10.1073/pnas.2019152118.
¹⁷ Kozhemyakina, E., et al. (2015). “Identification of a Prg4-expressing articular cartilage progenitor cell population in mice.” Arthritis Rheumatol. 67: 1261-1273.
¹⁸ Hellmuth, K., et al. (2008). “Specific inhibitors of the protein tyrosine phosphatase Shp2 identified by high-throughput docking.” Proc. Natl. Acad. Sci. USA 105(20): 7275-7280.
¹⁹ Grosskopf, S., et al. (2015). “Selective inhibitors of the protein tyrosine phosphatase SHP2 block cellular motility and growth of cancer cells in vitro and in vivo.” ChemMedChem 10(5): 815-826.
²⁰ Chen, L., et al. (2006). “Discovery of a novel Shp2 protein tyrosine phosphatase inhibitor.” Mol. Pharmacol. 70: 562-570.
²¹ Milne, G. W., et al. (1986). “The NCI drug information system. 2. DIS pre-registry.” J. Chem. Inf. Comput. Sci. 26: 159-168.
²² Lawrence, H. R., et al. (2008). “Inhibitors of Src homology-2 domain containing protein tyrosine phosphatase-2 (Shp2) based on oxindole scaffolds.” J. Med. Chem. 51(16): 4948-4956.
²³ Zhang, X., et al. (2010). “Salicylic acid based small molecule inhibitor for the oncogenic Src homology-2 domain containing protein tyrosine phosphatase-2 (SHP2).” J. Med. Chem. 53: 2482-2493.
²⁴ Zeng, L .- F., et al. (2014). “ Therapeutic Potential of Targeting the Oncogenic SHP2 Phosphatase.” J. Med. Chem. 57(15): 6594-6609.
²⁵ Chen, C., et al. (2015). “Discovery of a Novel Inhibitor of the Protein Tyrosine Phosphatase Shp2.” Scientific Reports 5: 17626.
²⁶ U.S. Pat. No. 10,590,090 entitled “2,5-disubstituted 3-methyl pyrazines and 2,5,6-trisubstituted 3-methyl pyrazines as allosteric SHP2 inhibitors.” Issued on Mar. 17, 2020 (Assignee: Revolution Medicines, Inc.).
²⁷ Chen, Y. N., et al. (2016). “Allosteric inhibition of SHP2 phosphatase inhibits cancers driven by receptor tyrosine kinases.” Nature 535: 148-152.
²⁸ Garcia Fortanet, J., et al. (2016). “Allosteric inhibition of SHP2: Identification of a potent, selective, and orally efficacious phosphatase inhibitor.” J. Med. Chem. 59(17): 7773-7782.
²⁹ LaMarche, M. J., et al. (2020). “Identification of TNO155, an Allosteric SHP2 Inhibitor for the Treatment of Cancer.” J. Med. Chem. 63: 22, 13578-13594.
³⁰ Fodor, M., et al. (2018). “Dual allosteric inhibition of SHP2 phosphatase.” ACS Chem. Biol. 13: 647e56. 31 Halgren T. (2009). “Identifying and characterizing binding sites and assessing druggability.” J. Chem. Inf. Model 49: 377e89.
³² Fodor, M., et al. (2018). “Dual allosteric inhibition of SHP2 phosphatase.” ACS Chem. Biol. 13: 647e56.
³³ Bagdanoff, J. T., et al. (2019). “Optimization of fused bicyclic allosteric SHP2 inhibitors.” J. Med. Chem. 62: 1781e92.
³⁴ Sarver, P., et al. (2019). “6-Amino-3-methylpyrimidinones as potent, selective, and orally efficacious SHP2 inhibitors.” J. Med. Chem. 62: 1793e802.
³⁵ Fodor, M., et al. (2018). “Dual allosteric inhibition of SHP2 phosphatase.” ACS Chem. Biol. 13: 647e56.
³⁶ Wu, et al. (2019). “Small molecule inhibitor that stabilizes the auto-inhibited conformation of the oncogenic tyrosine phosphatase SHP2.” J. Med. Chem. 62: 1125e37.
³⁷ Song, Z., et al. (2021). “Tyrosine phosphatase SHP2 inhibitors in tumor-targeted therapies.” Acta Pharmaceutica Sinica B, 11(1): 13-29.
³⁸ Nichols, R. J., et al. (2018). “RAS nucleotide cycling underlies the SHP2 phosphatase dependence of mutant BRAF-, NF1- and RAS-driven cancers.” Nat. Cell Biol. 20:1064e73.
³⁹ Koltun, E. S., et al. (2018). “RMC-4550, an allosteric inhibitor of SHP2: Synthesis, structure, and anti-tumor activity.” Cancer Research 78(13 Supplement): 4878.
⁴⁰ Sun, Y., et al. (2020). “Allosteric SHP2 Inhibitor, IACS-13909, Overcomes EGFR-Dependent and EGFR-Independent Resistance Mechanisms toward Osimertinib.” Cancer Res. 80: 4840-4853.
⁴¹ Yuting, S., et al. (2020). “Allosteric SHP2 Inhibitor, IACS-13909, Overcomes EGFR-Dependent and EGFR-Independent Resistance Mechanisms toward Osimertinib.” Cancer Res. 80(21): 4840-4853.
⁴² Sun, Y., et al. (2020). “Allosteric SHP2 Inhibitor, IACS-13909, Overcomes EGFR-Dependent and EGFR-Independent Resistance Mechanisms toward Osimertinib.” Cancer Res. 80(21): 4840-4853.
⁴³ Wang, M., et al. (2020). “Discovery of SHP2-D26 as a First, Potent, and Effective PROTAC Degrader of SHP2 Protein.” J. Med. Chem. 63(14): 7510-7528.
⁴⁴ Yang, W., et al. (2013). “Ptpn11 deletion in a novel progenitor causes metachondromatosis by inducing hedgehog signalling. Nature 499: 491-495.
⁴⁵ Decker, R. S., et al. (2017). “Cell origin, volume and arrangement are drivers of articular cartilage formation, morphogenesis and response to injury in mouse limbs.” Dev. Biol. 426: 56-68.
⁴⁶ Madisen, L., et al. (2010). “A robust and high-throughput Cre reporting and characterization system for the whole mouse brain.” Nature neuroscience 13: 133-140.
⁴⁷ Haseeb, A., et al. (2021). “SOX9 keeps growth plates and articular cartilage healthy by inhibiting chondrocyte dedifferentiation/osteoblastic redifferentiation.” Proc. Natl. Acad. Sci. USA 118(8): e2019152118; DOI: 10.1073/pnas.2019152118.
⁴⁸ Kozhemyakina, E., et al. (2015). “Identification of a Prg4-expressing articular cartilage progenitor cell population in mice.” Arthritis Rheumatol. 67: 1261-1273.
⁴⁹ Kumar, V., et al. (2015). ROBBINS AND COTRAN PATHOLOGIC BASIS OF DISEASE. (9^{th} ed.). p. 1182. ISBN 9780808924500.
⁵⁰ Kiani, C., et al. (2002). “Structure and Function of Aggrecan.” Cell Res. 12: 19-32.
⁵¹ Aigner, T. and Stove, J. (2003). “Collagens—major component of the physiological cartilage matrix, major target of cartilage degeneration, major tool in cartilage repair.” Adv. Drug Deliv. Rev. 55: 1569-1593.
⁵² Wang, L., et al. (2017). “SHP2 Regulates the Osteogenic Fate of Growth Plate Hypertrophic Chondrocytes.” Sci Rep 7: 12699.
⁵³ Glasson, S. S., et al. (2007). “The surgical destabilization of the medial meniscus (DMM) model of osteoarthritis in the 129/SvEv mouse.” Osteoarthritis Cartilage 15: 1061-1069.
⁵⁴ Glasson, S. S., et al. (2007). “The surgical destabilization of the medial meniscus (DMM) model of osteoarthritis in the 129/SvEv mouse.” Osteoarthritis Cartilage 15: 1061-1069.
⁵⁵ Sakamoto, K. M., et al. (2001). “Protacs: chimeric molecules that target proteins to the Skp1-Cullin-F box complex for ubiquitination and degradation.” Proc. Natl. Acad. Sci. USA 98(15): 8554-8559.
⁵⁶ Wang, M., et al. (2020). “Discovery of SHP2-D26 as a First, Potent, and Effective PROTAC Degrader of SHP2 Protein.” J. Med. Chem. 63: 7510-7528.
⁵⁷Id.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

What is claimed is:

1. A method of preventing and/or treating osteoarthritis (OA) in a subject at risk of, or afflicted with, OA by administrating a therapeutically effective amount of at least one SHP2 antagonistic agent to the subject.

2. The method of claim 1 wherein the at least one SHP2 antagonistic agent is selected from the group consisting of: a SHP2 inhibitor, a Proteolysis-Targeting Chimera (PROTAC) degrader of SHP2 proteins SHP2 (SHP2 degrader), and a SHP2 RNA interference (RNAi), or a SHP2 small interfering RNA (siRNA).

3. The method of claim 2 wherein the SHP2 inhibitor is a specific SHP2 inhibitor or an allosteric SHP2 inhibitor.

4. The method of claim 3 wherein the specific SHP2 inhibitor is selected from the group consisting of: PHPS1, GS-493, NSC-87877, NSC-117199, Ii-B08, Fumosorinone, and RMC-4630.

5. The method of claim 3 wherein the allosteric SHP2 inhibitor is selected from the group consisting of: SHP099, TNO-155, SHP-844, SHP-244, SHP-357, SHP-389, SHP-394, SHP-504, SHP-LY6, RMC-4550, IACS-13909, JAB-3068, JAB-3312, RLY-1971, and BBP-398.

6. The method of claim 2 wherein the SHP2 degrader is SHP2-D26.

7. The method of claim 2 wherein the SHP2 RNAi or SHP2 siRNA comprises SEQ ID NO: 1 and SEQ ID NO:2.

8. A method of preventing and/or treating osteoarthritis (OA) in a subject at risk of, or afflicted with, OA by administrating a therapeutically effective amount of a PROTAC degrader of SHP2 proteins (SHP2 degrader) to the subject.

9. The method of claim 8 wherein the SHP2 degrader is SHP2-D26.


SEQUENCE LISTING (2 sequences)

SEQ ID NO: 1 - DNA, 19 bp, Artificial Sequence
  Description of Artificial Sequence Synthetic oligonucleotide
atatggcggtccagcatta

SEQ ID NO: 2 - DNA, 19 bp, Artificial Sequence
  Description of Artificial Sequence Synthetic oligonucleotide
acactggtgattactatga